(19) United States
(12) Patent Application Publication
(10) Pub. No.: US 2013/0096347 A1
(43) Pub. Date: Apr. 18, 2013

(54) NOVEL PROCESS FOR PREPARING HIGHLY PURE TAPENTADOL OR A PHARMACEUTICALLY ACCEPTABLE SALT THEREOF

(71) Applicants: Mayur Devjibhai Khunt, Gujarat (IN); Sandipan Prabhurao Bondge, Maharashtra (IN); Nilesh Sudhir Patil, Maharashtra (IN); Haushabhau Shivaji Pagire, Maharashtra (IN); Nitin Sharadchandra Pradhan, Maharashtra (IN)

(72) Inventors: Mayur Devjibhai Khunt, Gujarat (IN); Sandipan Prabhurao Bondge, Maharashtra (IN); Nilesh Sudhir Patil, Maharashtra (IN); Haushabhau Shivaji Pagire, Maharashtra (IN); Nitin Sharadchandra Pradhan, Maharashtra (IN)

(73) Assignee: ACTAVIS GROUP PTC EHF, Hafnarfjordur (IS)

(21) Appl. No.: 13/638,055

(22) PCT Filed: Apr. 4, 2011
(86) PCT No.: PCT/IB2011/001484

§ 371 Date: Dec. 12, 2012

(30) Foreign Application Priority Data
Apr. 5, 2010 (IN) 936/CHE/2010

(51) Int. Cl. C07C 213/00; C07C 213/10; C07C 215/08; C07C 213/02; C07C 225/06

(52) U.S. Cl. CPC C07C 213/00; C07C 213/02; C07C 225/06; C07C 215/08; C07C 213/10
USPC 564/374; 241/23

(57) ABSTRACT

Provided herein is a novel, commercially viable and industrially advantageous process for the preparation of 3-[(1R,2R)-3-(dimethylamino)-1-ethyl-2-methylpropyl]phenol (Tapentadol), or a pharmaceutically acceptable salt thereof, and its intermediates, in high yield and purity. Provided also herein are novel solid state forms of tapentadol intermediates and processes for their preparation thereof. Provided further herein is a purification process for preparing highly pure tapentadol hydrochloride.

### CROSS REFERENCE TO RELATED APPLICATION

This application claims the benefit of priority to Indian provisional application No. 936/CHE/2010, filed on Apr. 5, 2010, which is incorporated herein by reference in its entirety.

### FIELD OF THE DISCLOSURE

Disclosed herein is a novel, commercially viable and industrially advantageous process for the preparation of 3-[(1R,2R)-3-(dimethylamino)-1-ethyl-2-methylpropyl]phenol (Tapentadol), or a pharmaceutically acceptable salt thereof, and its intermediates, in high yield and purity. Disclosed also herein are novel solid state forms of tapentadol intermediates and processes for their preparation thereof. Disclosed further herein is a purification process for preparing highly pure tapentadol hydrochloride.

### BACKGROUND

U.S. Pat. No. 6,248,737 reissued as U.S. RE39593 discloses a variety of 1-phenyl-3-dimethylaminopropane compounds, processes for their preparation, pharmaceutical compositions comprising the compounds, and methods of use thereof. These compounds have the utility as analgesic active ingredients in pharmaceutical compositions. Among them, Tapentadol hydrochloride, 3-[(1R,2R)-3-(dimethylamino)-1-ethyl-2-methylpropyl]phenol hydrochloride, is a centrally-acting analgesic with a unique dual mode of action as an agonist at the μ-opioid receptor and as a norepinephrine reuptake inhibitor. Tapentadol hydrochloride is represented by the following structural formula:

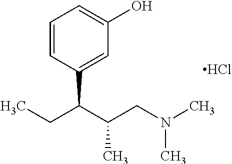 [SMILES: CC[C@@H](c1cccc(O)c1)[C@@H](C)CN(C)C ; Cl]

Various processes for the preparation of tapentadol, its enantiomers and related compounds, and their pharmaceutically acceptable salts are disclosed in U.S. Pat. Nos. U.S. RE39593 and 6,344,558; and PCT Publication Nos. WO 2004/108658, WO 2005/000788, WO 2008/012046, WO 2008/012047 and WO 2008/012283.

As per the process exemplified in example 25 of the U.S.RE39593 (hereinafter referred to as the '593 patent), (−)-(1R,2R)-3-(3-dimethylamino-1-ethyl-2-methylpropyl)-phenol hydrochloride is prepared by the reaction of (−)-(2S,3S)-1-dimethylamino-3-(3-methoxyphenyl)-2-methylpentan-3-ol hydrochloride with thionyl chloride to produce (−)-(2S,3S)-[3-chloro-3-(3-methoxyphenyl)-2-methylpentyl]-dimethylamine hydrochloride; followed by subsequent removal of the ‘Cl’ substituent by treatment with zinc borohydride, zinc cyanoborohydride or tin cyanoborohydride, to produce (−)-(2R,3R)-[3-(3-methoxyphenyl)-2-methylpentyl]-dimethylamine hydrochloride; which is then converted into (−)-(1R,2R)-3-(3-dimethylamino-1-ethyl-2-methylpropyl)-phenol hydrochloride by reaction with concentrated hydrobromic acid at reflux.

The synthesis of (2RS,3RS)-1-dimethylamino-3-(3-methoxyphenyl)-2-methylpentan-3-ol is described in the '593 patent. The separation of the diastereoisomers, that is the two enantiomeric pairs, is carried out by hydrochloride precipitation with trimethylchlorosilane/water in 2-butanone. The resolution of the racemic mixture of the two enantiomers of (2R,3R) and (2S,3S) configuration is carried by separation on a chiral HPLC column.

Methods involving column chromatographic separation of enantiomers on chiral stationary phases are generally undesirable for large-scale operations as they require additional expensive setup, adding to the cost of production, thereby making the processes commercially unfeasible.

U.S. Patent application No. 2008/0269524 discloses a resolution method for the separation of the two enantiomers from the enantiomeric pair, (2R,3R)/(2S,3S)-1-dimethylamino-3-(3-methoxyphenyl)-2-methylpentan-3-ol, with the aid of a chiral auxiliary, such as (+)-di-O,O′-p-toluyltartaric acid, (−)-di-O,O′-p-toluyltartaric acid and L-(+)-tartaric acid, in the presence of a suitable solvent such as 2-butanone.

U.S. Pat. No. 7,550,624 (hereinafter referred to as the '624 patent) discloses various pharmaceutically active salts and esters of 1-dimethylamino-3-(3-methoxyphenyl)-2-methylpentane-3-ol and 3-(3-dimethylamino-1-ethyl-1-hydroxy-2-methylpropyl)-phenol, and methods of using the same for treating or inhibiting increased urinary urgency or urinary incontinence and/or pain. The salts include ibuprofen, (S)-(+)-ibuprofen, (S)-(+)-naproxen, diclofenac, acetyl-salicylic acid, dipyron, indomethacin, ketoprofen, isoxicam, flurbiprofen, piroxicam and phenylbutazone. However, the '624 patent neither describes any resolution methods of the intermediates nor the use of any of the above salts for resolution processes.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in tapentadol or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities are limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure tapentadol or a pharmaceutically acceptable salt thereof substantially free of impurities, as well as processes for preparing thereof.

The processes for the preparation of tapentadol or a pharmaceutically acceptable salt thereof and its intermediates disclosed in the above mentioned prior art have the following disadvantages and limitations:
i) long reaction time periods, low yields and low purities of the products;
ii) the use of large volumes of solvents;
iii) the use of large volumes of concentrated hydrobromic acid (more than 23 volumes per 1 gm of the (−)-(2R,3R)-[3-(3-methoxyphenyl)-2-methylpentyl]-dimethylamine hydrochloride) in the demethylation reaction;
iv) the use of thionyl chloride for the chlorination reaction, which is toxic and dangerous to the environment; and
v) the overall processes generate a large quantity of chemical waste which is difficult to treat.

Based on the aforementioned drawbacks, the prior art processes have been found to be unsuitable for the preparation of tapentadol at lab scale and in commercial scale operations.

A need remains for an improved and commercially viable process of preparing tapentadol or a pharmaceutically acceptable salt thereof with high yields and high enantiomeric purity, to resolve the problems associated with the processes described in the prior art, and that will be suitable for large-scale preparation. Desirable process properties include non-hazardous conditions, environmentally friendly and easy to handle reagents, reduced reaction time periods, reduced cost, greater simplicity, increased purity, and increased yield of the product, thereby enabling the production of tapentadol and its pharmaceutically acceptable acid addition salts in high purity and in high yield.

### SUMMARY

In one aspect, provided herein is an efficient, industrially advantageous and environmentally friendly process for the preparation of 3-[(1R,2R)-3-(dimethylamino)-1-ethyl-2-methylpropyl]phenol of formula I (tapentadol) or a pharmaceutically acceptable salt thereof in high yield and with high chemical and enantiomeric purity. Moreover, the process disclosed herein involves non-hazardous and easy to handle reagents, reduced reaction time and reduced synthesis steps. The process avoids the tedious and cumbersome procedures of the prior processes and is convenient to operate on a commercial scale.

In another aspect, provided herein are solid state forms of tapentadol intermediates. In one aspect, the tapentadol intermediates in a crystalline form are provided. In yet another aspect, the tapentadol intermediates in an amorphous form are provided. It has also been found that the solid state forms of tapentadol intermediates are useful for preparing tapentadol or a pharmaceutically acceptable salt thereof, preferably tapentadol hydrochloride, in high purity.

In another aspect, encompassed herein is a purification process for obtaining highly pure tapentadol hydrochloride substantially free of impurities.

The process for the preparation of tapentadol or a pharmaceutically acceptable salt thereof disclosed herein has the following advantages over the processes described in the prior art:
i) the overall process involves reduced process steps and shorter reaction times;
ii) the process avoids the use of hazardous chemicals like hydrobromic acid, and tedious and cumbersome procedures like HBr distillation at post-reaction completion;
iii) the process involves the use of reduced and more appropriate volumes of the solvents;
iv) the process involves easy work-up methods and simple isolation processes; and
v) the overall yield and purities of the product are increased.

### BRIEF DESCRIPTION OF THE DRAWINGS

FIG. 1 is a characteristic powder X-ray diffraction (XRD) pattern of the solid state form of Racemic 3-(dimethylamino)-1-(3-hydroxyphenyl)-2-methylpropan-1-one hydrochloride prepared according to the Example 1.

FIG. 2 is a characteristic powder X-ray diffraction (XRD) pattern of the solid state form of Racemic 3-(dimethylamino)-1-(3-hydroxyphenyl)-2-methylpropan-1-one hydrochloride prepared according to the Example 2.

FIG. 3 is a characteristic powder X-ray diffraction (XRD) pattern of the solid state form of racemic mixture of (2R,3 S)/(2S,3R)-1-(Dimethylamino)-3-(3-hydroxyphenyl)-2-methylpentan-3-ol prepared according to the Example 5.

FIG. 4 is a characteristic powder X-ray diffraction (XRD) pattern of the solid state form of racemic mixture of (2R,3 S)/(2S,3R)-1-(Dimethylamino)-3-(3-hydroxyphenyl)-2-methylpentan-3-ol hydrochloride prepared according to the Example 4.

FIG. 5 is a characteristic powder X-ray diffraction (XRD) pattern of the solid state form of (2S,3R)-1-(Dimethylamino)-3-(3-hydroxyphenyl)-2-methylpentan-3-ol D-tartrate salt prepared according to the Example 6.

FIG. 6 is a characteristic powder X-ray diffraction (XRD) pattern of the solid state form of (2S,3R)-1-(Dimethylamino)-3-(3-hydroxyphenyl)-2-methylpentan-3-ol prepared according to the Example 7.

### DETAILED DESCRIPTION

According to one aspect, there is provided a process for preparing tapentadol, 3-[(1R,2R)-3-(dimethylamino)-1-ethyl-2-methylpropyl]phenol, of formula I:

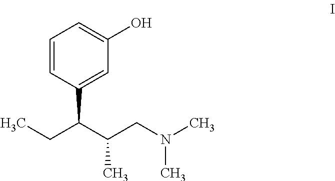 [SMILES: CC[C@@H](c1cccc(O)c1)[C@@H](C)CN(C)C]

or a pharmaceutically acceptable salt thereof, comprising
a) reacting 3-hydroxypropiophenone of formula V:

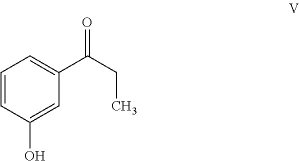

with N,N-dimethylamine hydrochloride in the presence of para-formaldehyde in a first solvent to produce a racemic 3-(dimethylamino)-1-(3-hydroxyphenyl)-2-methylpropan-1-one of formula IV:

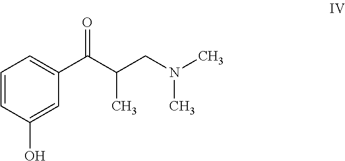

or an acid addition salt thereof;
b) reacting the compound of formula IV or the acid addition salt thereof with an ethyl magnesium halide in a second solvent to produce a racemic mixture of (2R,3S)/(2S,3R)-1-(Dimethylamino)-3-(3-hydroxyphenyl)-2-methylpentan-3-ol of formula III:

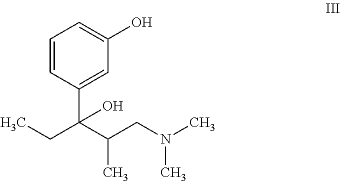 [SMILES: CCC(O)(c1cccc(O)c1)C(C)CN(C)C]

or an acid addition salt thereof;
c) treating the racemic mixture obtained in step-(b) with an optically active acid in a third solvent to produce a diastereomeric mixture containing a diastereomeric excess of a desired diastereomeric salt;
d) separating the desired diastereomeric salt from the diastereomeric mixture obtained in step-(c);
e) neutralizing the desired diastereomeric salt obtained in step-(d) with a base in a fourth solvent to produce an enantiomerically pure (2S,3R)-1-(Dimethylamino)-3-(3-hydroxyphenyl)-2-methylpentan-3-ol of formula II:

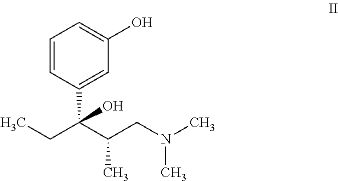 [SMILES: CC[C@](O)(c1cccc(O)c1)[C@@H](C)CN(C)C]

and optionally converting the enantiomerically pure compound of formula II obtained into an acid addition salt thereof; and
f) reacting the enantiomerically pure compound of formula II or the acid addition salt thereof obtained in step-(e) with trifluoroacetic anhydride in a fifth solvent to produce a reaction mass, followed by hydrogenating the reaction mass in the presence of a hydrogenation catalyst to produce the tapentadol of formula I, and optionally converting the tapentadol of formula I obtained into a pharmaceutically acceptable salt thereof.

Exemplary first solvents used in step-(a) include, but are not limited to, water, an alcohol, an ether, a hydrocarbon, a chlorinated hydrocarbon, a nitrile solvent, a polar aprotic solvent, and mixtures thereof. The term solvent also includes mixtures of solvents.

In one embodiment, the first solvent is selected from the group consisting of water, methanol, ethanol, n-propanol, isopropanol, n-butanol, isobutanol, tert-butanol, amyl alcohol, acetonitrile, dichloromethane, dichloroethane, chloroform, carbon tetrachloride, tetrahydrofuran, 2-methyl tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically, the first solvent is selected from the group consisting of methanol, ethanol, isopropanol, n-butanol, isobutanol, tert-butanol, amyl alcohol, acetonitrile, and mixtures thereof; and a most specific first solvent is isopropanol.

In another embodiment, the reaction in step-(a) is carried out at a temperature of 0° C. to the reflux temperature of the solvent used for at least 4 hours, specifically at a temperature of about 25° C. to the reflux temperature of the solvent used for about 5 hours to about 20 hours, and more specifically at the reflux temperature of the solvent used for about 8 hours to about 12 hours.

As used herein, the term “reflux temperature” means the temperature at which the solvent or solvent system refluxes or boils at atmospheric pressure.

In one embodiment, the N,N-dimethylamine hydrochloride is used in a ratio of about 1 to 4 equivalents, specifically about 2 to 2.5 equivalents, with respect to the 3-hydroxypropiophenone of formula V in order to ensure a proper course of the reaction.

The reaction mass containing the racemic 3-(dimethylamino)-1-(3-hydroxyphenyl)-2-methylpropan-1-one of formula IV obtained in step-(a) may be subjected to usual work up such as a washing, an extraction, a pH adjustment, an evaporation or a combination thereof. The reaction mass may be used directly in the next step to produce the racemic mixture of (2R,3S)/(2S, 3R)-1-(Dimethylamino)-3-(3-hydroxyphenyl)-2-methylpentan-3-ol of formula III, or the racemic 3-(dimethylamino)-1-(3-hydroxyphenyl)-2-methylpropan-1-one of formula IV may be isolated and then used in the next step.

In one embodiment, the racemic 3-(dimethylamino)-1-(3-hydroxyphenyl)-2-methylpropan-1-one of formula IV is isolated from a suitable solvent by conventional methods such as cooling, seeding, partial removal of the solvent from the solution, by adding an anti-solvent to the solution, evaporation, vacuum distillation, or a combination thereof

In another embodiment, the solvent used to isolate the racemic 3-(dimethylamino)-1-(3-hydroxyphenyl)-2-methylpropan-1-one of formula IV is selected from the group consisting of water, an aliphatic ether, a hydrocarbon solvent, a chlorinated hydrocarbon, and mixtures thereof. Specifically, the solvent is selected from the group consisting of water, dichloromethane, diethyl ether, diisopropyl ether, n-heptane, n-pentane, n-hexane, cyclohexane, and mixtures thereof. A most specific solvent is dichloromethane.

In another embodiment, the reaction mass containing the racemic 3-(dimethylamino)-1-(3-hydroxyphenyl)-2-methylpropan-1-one of formula IV obtained in step-(a) may be subjected to usual work up as described above and then converted into its acid addition salt by reacting with a suitable acid in a suitable solvent, wherein the solvent is selected from the group consisting of water, an alcohol, a ketone, an ether, a hydrocarbon, a chlorinated hydrocarbon, a nitrile solvent, a polar aprotic solvent, and mixtures thereof

Exemplary acids used for preparing the acid addition salts of the compound of formula IV include, but are not limited to, hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, and tartaric acid. The salt derived from hydrochloric acid is particularly preferred.

In one embodiment, the reaction mass containing the racemic 3-(dimethylamino)-1-(3-hydroxyphenyl)-2-methylpropan-1-one of formula IV obtained after completion of the reaction is cooled to ambient temperature, diluted with water and washed with a suitable solvent selected from group consisting of a hydrocarbon, a chlorinated hydrocarbon, an ester, an ether, and mixtures thereof. Specific solvent is toluene or dichloromethane. The resulting aqueous layer is separated and basified with a base to adjust the pH to 7 to 14, specifically to 9 to 10, followed by extraction with a suitable organic solvent selected from the group as described above. The base can be an organic or inorganic base, specifically inorganic base and more specifically aqueous ammonia. The separated aqueous layer is optionally extracted with the organic solvent selected from the group as described above, followed by washing the combined organic layer with water and then drying over sodium sulfate, prior to the solvent removal, to obtain the compound of formula IV.

In another embodiment, the racemic 3-(dimethylamino)-1-(3-hydroxyphenyl)-2-methylpropan-1-one of formula IV formed in step-(a) is isolated as a solid in the form of its hydrochloride salt.

According to another aspect, there is provided solid state forms of racemic 3-(dimethylamino)-1-(3-hydroxyphenyl)-2-methylpropan-1-one hydrochloride salt.

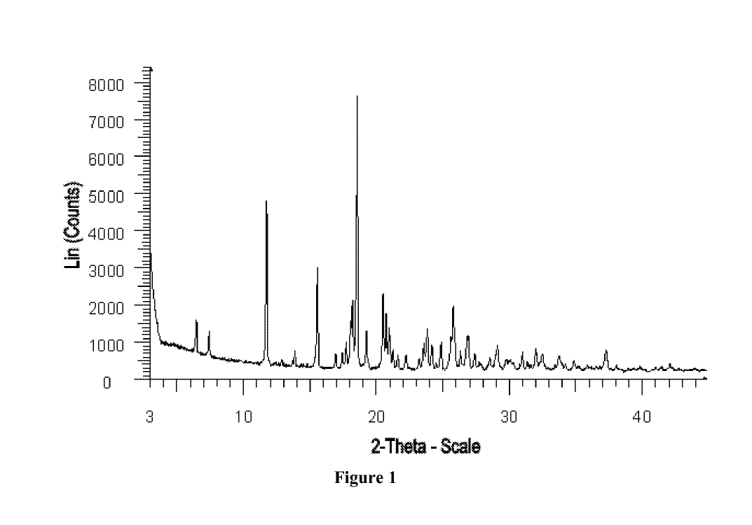

In one embodiment, the solid state form of the racemic 3-(dimethylamino)-1-(3-hydroxyphenyl)-2-methylpropan-1-one hydrochloride, obtained by the process exemplified in example 1 disclosed herein, is a crystalline form characterized by a powder X-ray diffraction pattern having peaks at about 6.38, 7.34, 11.68, 13.81, 15.52, 16.9, 17.42, 17.67, 18.14, 18.51, 19.20, 20.47, 20.70, 20.96, 21.23, 21.58, 22.20, 23.55, 23.80, 24.16, 24.85, 25.53, 25.77, 26.31, 26.85, 27.39, 29.09, 30.99, 32.49 and 33.76±0.2 degrees 2-theta substantially in accordance with FIG. 1.

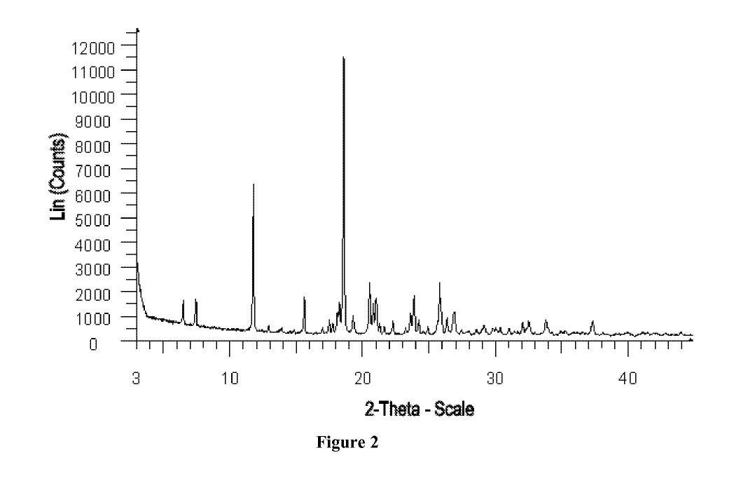

In another embodiment, the solid state form of the racemic 3-(dimethylamino)-1-(3-hydroxyphenyl)-2-methylpropan-1-one hydrochloride, obtained by the process exemplified in example 2 disclosed herein, is a crystalline form characterized by a powder X-ray diffraction pattern having peaks at about 6.42, 7.38, 11.71, 15.57, 18.19, 18.55, 19.26, 20.51, 20.77, 21.0, 22.25, 23.59, 23.85, 24.20, 25.81, 26.35, 26.87, 32.54 and 33.81±0.2 degrees 2-theta substantially in accordance with FIG. 2.

In one embodiment, the ethyl magnesium halide used in step-(b) is selected from the group consisting of ethyl magnesium chloride, ethyl magnesium bromide and ethyl magnesium iodide. A specific ethyl magnesium halide is ethyl magnesium chloride.

Exemplary second solvents used in step-(b) include, but are not limited to, a ketone, a cyclic ether, an aliphatic ether, and mixtures thereof. The term solvent also includes mixtures of solvents.

In one embodiment, the second solvent is selected from the group consisting of acetone, methyl ethyl ketone, methyl isobutyl ketone, methyl tert-butyl ketone, tetrahydrofuran, 2-methyl tetrahydrofuran, dioxane, diethyl ether, diisopropyl ether, monoglyme, diglyme, and mixtures thereof; and a most specific second solvent is tetrahydrofuran or 2-methyl tetrahydrofuran.

In another embodiment, the reaction in step-(b) is carried out at a temperature of below about 60° C. for at least 30 minutes, specifically at a temperature of about 0° C. to about 25° C. for about 1 hour to about 10 hours, and more specifically at about 0° C. to about 15° C. for about 2 hours to about 4 hours. In another embodiment, the reaction mass may be quenched with aqueous solution of ammonium chloride and water after completion of the reaction.

In one embodiment, the ethyl magnesium halide in step-(b) is used in a ratio of about 2 to 20 equivalents, specifically about 3 to 10 equivalents, with respect to the racemic 3-(dimethylamino)-1-(3-hydroxyphenyl)-2-methylpropan-1-one formula IV in order to ensure a proper course of the reaction.

The reaction mass containing the racemic mixture of (2R,3S)/(2S,3R)-1-(Dimethylamino)-3-(3-hydroxyphenyl)-2-methylpentan-3-ol of formula III obtained in step-(b) may be subjected to usual work up methods as described above. The reaction mass may be used directly in the next step or the racemic mixture of (2R,3S)/(2S,3R)-1-(Dimethylamino)-3-(3-hydroxyphenyl)-2-methylpentan-3-ol of formula III may be isolated and then used in the next step.

In one embodiment, the racemic mixture of (2R,3S)/(2S,3R)-1-(Dimethylamino)-3-(3-hydroxyphenyl)-2-methylpentan-3-ol of formula III is isolated from a suitable solvent by the methods as described above.

The solvent used to isolate the racemic mixture of (2R,3S)/(2S,3R)-1-(Dimethylamino)-3-(3-hydroxyphenyl)-2-methylpentan-3-ol of formula III is selected from the group consisting of water, an aliphatic ether, a hydrocarbon solvent, a chlorinated hydrocarbon, and mixtures thereof. Specifically, the solvent is selected from the group consisting of water, dichloromethane, diethyl ether, diisopropyl ether, n-heptane, n-pentane, n-hexane, cyclohexane, and mixtures thereof. A most specific solvent is dichloromethane.

In another embodiment, the reaction mass containing the racemic mixture of (2R,3 S)/(2S,3R)-1-(Dimethylamino)-3-(3-hydroxyphenyl)-2-methylpentan-3-ol of formula III obtained in step-(b) may be subjected to usual work up as described above and then converted into its acid addition salt by reacting with a suitable acid in a suitable solvent, wherein the solvent is selected from the group consisting of water, an alcohol, a ketone, an ether, a hydrocarbon, a chlorinated hydrocarbon, a nitrile solvent, a polar aprotic solvent, and mixtures thereof. Specific solvents are alcohols and more specifically isopropanol.

The acid used for preparing the acid addition salts of the compound of formula III is selected from the group as described above. The salt derived from hydrochloric acid is particularly preferred.

In one embodiment, the reaction mass obtained after completion of the reaction is quenched with aqueous solution of ammonium chloride and water, separated the organic layer and the aqueous layer is optionally extracted with an organic solvent. The resulting organic layer is washed with water, followed by drying over sodium sulfate, prior to the removal of the solvent, to obtain the free base (solid) of compound of formula III. The solvent used for the extraction is selected from the group consisting of a hydrocarbon, a chlorinated hydrocarbon, an ester, an ether, and mixtures thereof; specifically a chlorinated hydrocarbon; and more specifically dichloromethane.

In another embodiment, the racemic mixture of (2R,3S)/(2S,3R)-1-(Dimethylamino)-3-(3-hydroxyphenyl)-2-methylpentan-3-ol of formula III formed in step-(b) is isolated as a solid in the form of its hydrochloride salt.

According to another aspect, there is provided a solid state form of racemic mixture of (2R,3 S)/(2S, 3R)-1-(Dimethylamino)-3-(3-hydroxyphenyl)-2-methylpentan-3-ol of formula III.

According to another aspect, there is provided a solid state form of racemic mixture of (2R,3 S)/(2S, 3R)-1-(Dimethylamino)-3-(3-hydroxyphenyl)-2-methylpentan-3-ol hydrochloride salt.

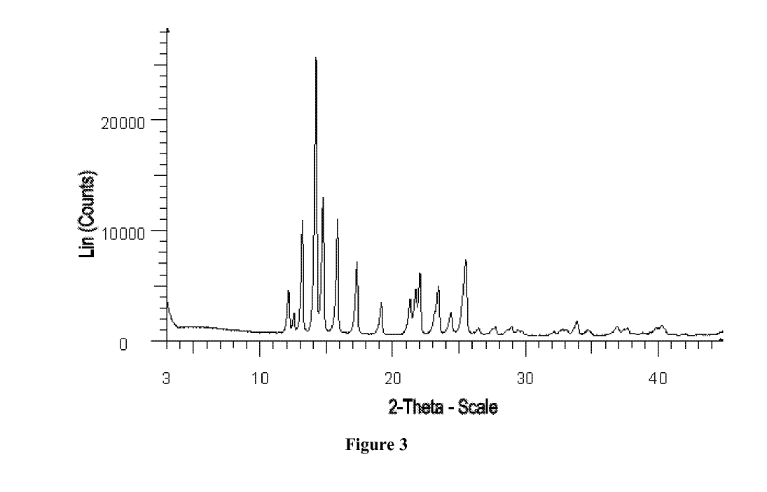

In one embodiment, the solid state form of the racemic mixture of (2R,3 S)/(2S,3R)-1-(Dimethylamino)-3-(3-hydroxyphenyl)-2-methylpentan-3-ol, obtained by the process exemplified in example 5 disclosed herein, is a crystalline form characterized by a powder X-ray diffraction pattern having peaks at about 12.1, 12.51, 13.13, 14.16, 14.7, 15.78, 17.25, 19.09, 21.28, 21.69, 22.01, 23.10, 23.4 and 24.36±0.2 degrees 2-theta substantially in accordance with FIG. 3.

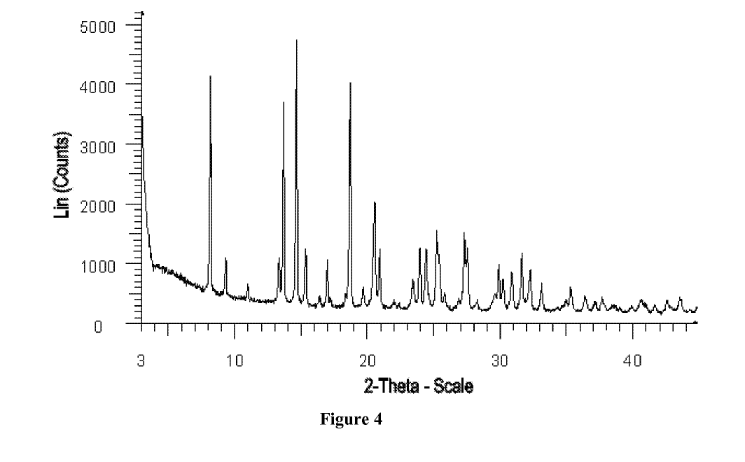

In another embodiment, the solid state form of the racemic mixture of (2R,3 S)/(2S,3R)-1-(Dimethylamino)-3-(3-hydroxyphenyl)-2-methylpentan-3-ol hydrochloride, obtained by the process exemplified in example 4 disclosed herein, is a crystalline form characterized by a powder X-ray diffraction pattern having peaks at about 8.11, 9.27, 10.92, 13.30, 13.64, 14.62, 15.30, 18.32, 18.66, 19.65, 20.51, 20.90, 23.42, 23.93, 24.41, 25.25, 25.80, 27.32, 27.52, 29.59, 29.91, 30.22, 30.88, 31.63, 32.26, 35.34 and 36.43±0.2 degrees 2-theta substantially in accordance with FIG. 4.

The term “enantiomerically pure (2S,3R)-1-(Dimethylamino)-3-(3-hydroxyphenyl)-2-methylpentan-3-ol of formula II” refers to the compound of formula II having enantiomeric purity greater than about 95%, specifically greater than about 98%, more specifically greater than about 99.5%, and most specifically greater than about 99.9% measured by HPLC.

Exemplary optically active acids used in step-(c) include, but are not limited to, optically active tartaric acid, optically active di-aroyl-tartaric acid, (αS)-6-methoxy-α-methyl-2-naphthaleneacetic acid (S-naproxen), malic acid, mandelic acid and its derivatives, and camphorsulphonic acid and its derivatives.

In one embodiment, the optically active acid is selected from the group consisting of D-(−)-tartaric acid, L-(+)-tartaric acid, S-naproxen, (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof. A most specific optically active acid is D-(−)-tartaric acid.

The optically active acid in step-(c) can be optionally used as a mixture with other acids (adjuvant acids) that can be organic or inorganic, such as hydrochloric acid, p-toluensulphonic acid, methanosulphonic acid or a mixture thereof, in molar proportions that vary between 0.5% and 50% (this molar percentage refers to the total of the mixture of the chiral acid and the adjuvant acid).

In one embodiment, the resolving agent in step-(c) is used in a ratio of about 1 to 5 equivalents, specifically about 1 to 1.5 equivalents, with respect to the racemic mixture of (2R,3S)/(2S,3R)-1-(Dimethylamino)-3-(3-hydroxyphenyl)-2-methylpentan-3-ol of formula III.

Exemplary third solvents used in step-(c) include, but are not limited to, water, an alcohol, a ketone, an ester, a hydrocarbon, a chlorinated hydrocarbon, a polar aprotic solvent, and mixtures thereof. The term solvent also includes mixtures of solvents.

In one embodiment, the third solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, n-pentane, n-hexane, n-heptane, cyclohexane, toluene, xylene, N,N-dimethylformamide, N,N-dimethylacetamide, dimethylsulfoxide, and mixtures thereof; more specifically, the third solvent is selected from the group consisting of water, methanol, ethanol, isopropanol, and mixtures thereof; and most specifically a mixture of water and isopropanol.

In another embodiment, the reaction in step-(c) is carried out at a temperature of 0° C. to the reflux temperature of the solvent used for at least 30 minutes, specifically at a temperature of about 50° C. to about 100° C. for about 30 minutes to about 10 hours, and more specifically at a temperature of about 60° C. to about 90° C. for about 1 hour to about 5 hours.

The term “diastereomeric excess” refers to formation of a diastereomer having one configuration at chiral carbon in excess over that having the opposite configuration. Specifically, one diastereomer is formed in above about 60% of the mixture of diastereomers over the other, and more specifically above about 80% of the mixture of diastereomers.

The separation of diastereomers in step-(d) may be required to provide stereomers with desired optical purity. It is well known that diastereomers differ in their properties such as solubility, and thus can be separated based on the differences in their properties. The separation of the diastereomers can be performed using the methods known to the person skilled in the art. These methods include chromatographic techniques and fractional crystallization, and a preferable method is fractional crystallization.

In one embodiment, a solution of the diastereomeric mixture is subjected to fractional crystallization. The solution of the diastereomeric mixture may be a solution of the reaction mixture obtained as above or a solution prepared by dissolving the isolated diastereomeric mixture in a solvent. Specific solvents used for the separation include, but are not limited to, water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof more specifically, the solvent is selected from the group consisting of water, methanol, ethanol, isopropanol, acetonitrile, and mixtures thereof and most specifically water, isopropanol, and mixtures thereof.

The fractional crystallization of one diastereomer from the solution of the diastereomeric mixture can be performed by conventional methods such as cooling, partial removal of solvents, using anti-solvent, seeding, or a combination thereof

Fractional crystallization can be repeated until the desired chiral purity is obtained. In general, usually one or two crystallizations may be sufficient.

In one embodiment, the base used in step-(e) is an organic or inorganic base. Specific organic bases are triethylamine, trimethylamine, dimethyl amine and tert-butyl amine.

In another embodiment, the base is an inorganic base. Exemplary inorganic bases include, but are not limited to, hydroxides, alkoxides, bicarbonates and carbonates of alkali or alkaline earth metals; and ammonia. Specific inorganic bases are aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide; and more specifically ammonia, sodium hydroxide, potassium hydroxide, sodium carbonate and potassium carbonate.

Exemplary fourth solvents used in step-(e) include, but are not limited to, water, an alcohol, a ketone, a cyclic ether, an aliphatic ether, a hydrocarbon, a chlorinated hydrocarbon, a nitrile, an ester, and mixtures thereof. The term solvent also includes mixtures of solvents.

In one embodiment, the fourth solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, and mixtures thereof more specifically, the fourth solvent is selected from the group consisting of water, methylene chloride, n-hexane, n-heptane, cyclohexane, toluene, xylene, and mixtures thereof; and most specifically a mixture of water and methylene chloride.

In one embodiment, the pH of the reaction mass in step-(e) is adjusted to above 7, and specifically between 7 and 8.

The reaction mass containing the enantiomerically pure (2S,3R)-1-(Dimethylamino)-3-(3-hydroxyphenyl)-2-methylpentan-3-ol of formula II obtained in step-(e) may be subjected to usual work up methods as described above, followed by isolation from a suitable organic solvent by the methods such as cooling, partial removal of the solvent from the solution, addition of precipitating solvent, or a combination thereof

In another embodiment, the acid addition salt of (2S,3R)-1-(Dimethylamino)-3-(3-hydroxyphenyl)-2-methylpentan-3-ol of formula II is derived from a therapeutically acceptable acid selected from the group as described above.

In another embodiment, the (2S,3R)-1-(Dimethylamino)-3-(3-hydroxyphenyl)-2-methylpentan-3-ol D-tartrate salt formed in step-(d) is isolated as a solid.

According to another aspect, there is provided a solid state form of (2S,3R)-1-(Dimethylamino)-3-(3-hydroxyphenyl)-2-methylpentan-3-ol D-tartrate salt.

According to another aspect, there is provided a solid state form of (2S,3R)-1-(Dimethylamino)-3-(3-hydroxyphenyl)-2-methylpentan-3-ol of formula II.

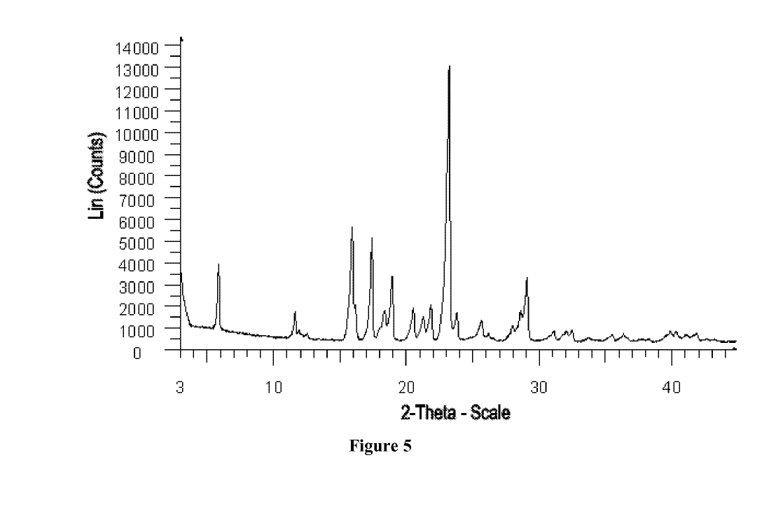

In one embodiment, the solid state form of the (2S,3R)-1-(Dimethylamino)-3-(3-hydroxyphenyl)-2-methylpentan-3-ol D-tartrate salt, obtained by the process exemplified in example 5 disclosed herein, is a crystalline form characterized by a powder X-ray diffraction pattern having peaks at about 5.78, 11.55, 15.86, 16.14, 17.35, 18.33, 18.88, 20.48, 21.24, 21.81, 23.17, 23.77, 28.59 and 29.07±0.2 degrees 2-theta substantially in accordance with FIG. 5.

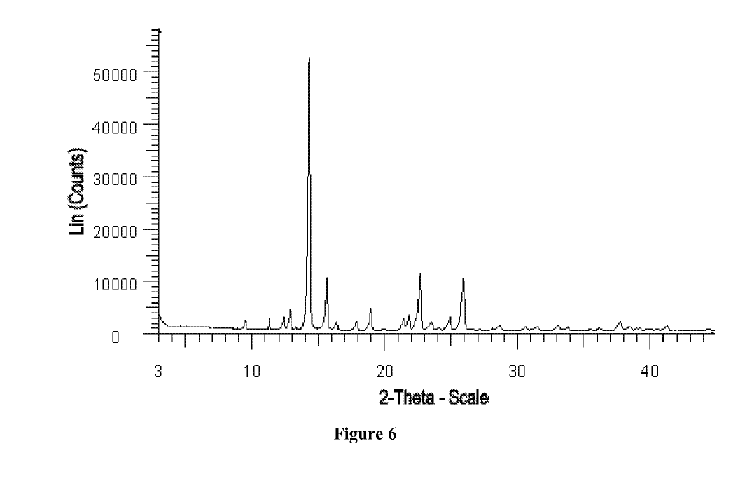

In another embodiment, the solid state form of the (2S,3R)-1-(Dimethylamino)-3-(3-hydroxyphenyl)-2-methylpentan-3-ol of formula II, obtained by the process exemplified in example 4 disclosed herein, is a crystalline form characterized by a powder X-ray diffraction pattern having peaks at about 9.44, 11.25, 12.35, 12.83, 14.27, 15.6, 16.34, 17.85, 18.93, 21.42, 21.81, 22.63, 23.48, 24.91 and 25.92±0.2 degrees 2-theta substantially in accordance with FIG. 6.

In one embodiment, the compound of formula III can be resolved into its another isomer (undesired isomer) using the same reaction conditions described above in the presence of an optically active acid like L-(+)-tartaric acid as a resolving agent.

Exemplary fifth solvents used in step-(f) include, but are not limited to, water, an alcohol, a ketone, a cyclic ether, an aliphatic ether, a hydrocarbon, a chlorinated hydrocarbon, a nitrile solvent, and mixtures thereof. The term solvent also includes mixtures of solvents.

In one embodiment, the fifth solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, dichloromethane, dichloroethane, chloroform, carbon tetrachloride, tetrahydrofuran, 2-methyl tetrahydrofuran, dioxane, diethyl ether, diisopropyl ether, monoglyme, diglyme, n-pentane, n-hexane, n-heptane, cyclohexane, toluene, xylene, and mixtures thereof; more specifically, the fifth solvent is selected from the group consisting of methanol, ethanol, isopropanol, acetonitrile, tetrahydrofuran, 2-methyl tetrahydrofuran, and mixtures thereof; and a most specific solvent is 2-methyl tetrahydrofuran.

In another embodiment, the reaction in step-(f) is carried out at a temperature of −20° C. to the reflux temperature of the solvent used for at least 10 minutes, specifically at a temperature of about 0° C. to about 50° C. for about 20 minutes to about 6 hours, and more specifically at a temperature of about 0° C. to about 35° C. for about 1 hour to about 4 hours.

Exemplary hydrogenation catalysts used in step-(f) include, but are not limited to, palladium hydroxide, palladium on carbon, platinum on carbon, platinum oxide, rhodium on carbon, and rhodium on alumina. A specific hydrogenation catalyst is palladium on carbon.

In one embodiment, the hydrogenation reaction in step-(f) is carried out at a temperature of 0° C. to the reflux temperature of the solvent used for at least 30 minutes, specifically at a temperature of about 20° C. to about 65° C. for about 45 minutes to about 7 hours, and more specifically at about 50° C. to about 60° C. for about 1 hour to about 5 hours.

In one embodiment, the hydrogenation reaction in step-(b) is carried out under hydrogen pressure or in the presence of a hydrogen transfer reagent, specifically under hydrogen pressure.

Exemplary hydrogen transfer reagents include, but are not limited to, formic acid, salts of formic acid such as ammonium formate, sodium formate, trialkyl ammonium formates, hydrazine, 1,3-cyclohexadiene, 1,4-cyclohexadiene and cyclohexene.

As used herein, the term ‘alkyl’ means saturated, acyclic groups which may be straight or branched containing from one to about seven carbon atoms as exemplified by methyl, ethyl, propyl, isopropyl, butyl, hexyl or heptyl.

Specific hydrogen transfer reagents are formic acid, ammonium formate, sodium formate, trimethylammonium formate and tributylammonium formate; and more specifically ammonium formate.

In another embodiment, the hydrogenation catalyst is used in the ratio of about 0.05% (w/w) to 10% (w/w), specifically about 0.5% (w/w) to 2.5% (w/w), with respect to (−)-3-[(1R,2R)-3-(dimethylamino)-1-ethyl-1-hydroxy-2-methylpropyl]phenol of formula II in order to ensure a proper course of the reaction.

The reaction mass containing the tapentadol of formula I obtained in step-(f) may be subjected to usual work up methods as described above.

In one embodiment, the tapentadol of formula I formed in step-(f) is isolated from a suitable solvent by the methods as described above.

In one embodiment, the reaction mass containing compound of formula I obtained in step-(f) is filtered to remove the catalyst, and the filtrate is distilled at about 0° C. to about 65° C., preferably at 55° C., under vacuum to obtain a residue. Water and ammonia are added to the residue and the resulting aqueous solution is extracted with organic solvent, wherein the organic solvent is selected from the group consisting of a hydrocarbon, a chlorinated hydrocarbon, an ester, an ether, and mixtures thereof; preferably a chlorinated hydrocarbon; and more preferably dichloromethane. The combined organic layer is washed with water, followed by drying over sodium sulfate prior to removal of solvent under reduced pressure to obtain the tapentadol of formula I.

Pharmaceutically acceptable salts of tapentadol can be prepared in high purity by using the highly pure tapentadol obtained by the method disclosed herein, by known methods.

Specific pharmaceutically acceptable salts of tapentadol include, but are not limited to, hydrochloride, hydrobromide, oxalate, nitrate, sulphate, phosphate, fumarate, succinate, maleate, besylate, tosylate, palmitate and tartrate; and more specifically hydrochloride.

The solvent used to isolate the tapentadol of formula I or a pharmaceutically acceptable acid addition salt thereof is selected from the group comprising water, alcohols, chlorinated hydrocarbons, hydrocarbons, ketones, nitriles, esters, ethers, polar aprotic solvents, and mixtures thereof. Specifically, the solvent is selected from the group consisting of water, acetone, methanol, ethanol, n-propanol, isopropanol, ethyl acetate, dichloromethane, n-pentane, n-hexane, n-heptane, cyclohexane, toluene and mixture thereof; and a more specific solvent is isopropyl alcohol.

In one embodiment, the tapentadol hydrochloride obtained by the process described above can be further optionally purified by the purification process disclosed herein after.

The highly pure tapentadol or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

In another embodiment, the highly pure tapentadol or a pharmaceutically acceptable salt thereof obtained by the process disclosed herein has a total purity, includes both chemical and enantiomeric purity, of greater than about 99%, specifically greater than about 99.5%, more specifically greater than about 99.9%, and most specifically greater than about 99.95% as measured by HPLC. For example, the purity of the highly pure tapentadol or a pharmaceutically acceptable salt thereof is about 99% to about 99.9%, or about 99.5% to about 99.99%.

It has been found that the following three isomers of tapentadol (Isomer-A, Isomer-B and Isomer-D) are formed as impurities in the synthesis of tapentadol hydrochloride and they remain in the final product:
i) Isomer-A: 3- [(1R,2S)-3-(dimethylamino)-1-ethyl-2-methylpropyl]phenol hydrochloride ((R,S)-tapentadol hydrochloride);
ii) Isomer-B: 3- [(1S,2R)-3-(dimethylamino)-1-ethyl-2-methylpropyl]phenol hydrochloride ((S,R)-tapentadol hydrochloride);
iii) Isomer-D: 3-[(1S,2S)-3-(dimethylamino)-1-ethyl-2-methylpropyl]phenol hydrochloride ((S,S)-tapentadol hydrochloride).

In addition to the above impurities, there are five other impurities identified at 1.09, 1.14, 1.24, 1.47 and 2.19 ±0.02 RRt's (hereinafter referred to as the ‘1.09 RRt’ impurity, ‘1.14 RRt’ impurity, ‘1.24 RRt’ impurity, ‘1.47 RRt’ impurity and ‘2.19 RRt’ impurity, collectively referred to as the ‘single maximum unknown impurities’), whose presence is observed in tapentadol hydrochloride.

Regarding the specific RRt values of impurities disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.02.

Thus there is a need for a method for determining the level of impurities in tapentadol hydrochloride samples and removing the impurities.

Extensive experimentation has been carried out by the present inventors to reduce the level of the isomer-A, isomer-B and isomer-D, and ‘1.09 RRt’, ‘1.14 RRt’, ‘1.24 RRt’, ‘1.47 RRt’ and ‘2.19 RRt’ impurities in tapentadol hydrochloride. As a result, it has been found that the isomer-A, isomer-B and isomer-D, and ‘1.09 RRt’, ‘1.14 RRt’, ‘1.24 RRt’, ‘1.47 RRt’ and ‘2.19 RRt’ impurities formed in the preparation of the tapentadol hydrochloride can be reduced or completely removed by the purification process disclosed herein.

According to another aspect, there is provided a highly pure tapentadol hydrochloride substantially free of at least one, or more, of the isomer-A, isomer-B and isomer-D, and ‘1.09 RRt’, ‘1.14 RRt’, ‘1.24 RRt’, ‘1.47 RRt’ and ‘2.19 RRt’ impurities.

According to another aspect, there is provided a purification process for obtaining highly pure tapentadol hydrochloride substantially free of impurities, comprising:
a) providing a solution of tapentadol hydrochloride in a solvent or a solvent mixture at a temperature of above 40° C., wherein the solvent or the solvent mixture is selected from the group consisting of water, methanol, ethanol, isopropyl alcohol, acetonitrile, and mixtures thereof;
b) optionally, subjecting the solution obtained in step-(a) to carbon treatment or silica gel treatment; and
c) isolating and recovering the highly pure tapentadol hydrochloride substantially free of impurities from the solution obtained in step-(a) or step-(b), wherein the isolation is carried out by cooling the solution at a temperature of below 40° C.

As used herein, “highly pure tapentadol hydrochloride substantially free of impurities” refers to tapentadol hydrochloride comprising one, or more, of the isomer-A, isomer-B and isomer-D, and ‘1.09 RRt’, ‘1.14 RRt’, ‘1.24 RRt’, ‘1.47 RRt’ and ‘2.19 RRt’ impurities, each one, in an amount of less than about 0.15 area-% as measured by HPLC. Specifically, the tapentadol hydrochloride, as disclosed herein, contains less than about 0.1 area-%, more specifically less than about 0.05 area-%, still more specifically less than about 0.02 area-% of one, or more, of the isomer-A, isomer-B and isomer-D, and ‘1.09 RRt’, ‘1.14 RRt’, ‘1.24 RRt’, ‘1.47 RRt’ and ‘2.19 RRt’ impurities, and most specifically is essentially free of one, or more, of the isomer-A, isomer-B and isomer-D, and ‘1.09 RRt’, ‘1.14 RRt’, ‘1.24 RRt’, ‘1.47 RRt’ and ‘2.19 RRt’ impurities.

Step-(a) of providing a solution of tapentadol hydrochloride includes dissolving tapentadol hydrochloride in the solvent or the solvent mixture, or obtaining an existing solution from a previous processing step.

In one embodiment, the tapentadol hydrochloride is dissolved in the solvent or the solvent mixture at a temperature of about 50° C. to the reflux temperature of the solvent used, specifically at about 60° C. to about 90° C., and more specifically at about 70° C. to about 80° C.

As used herein, “reflux temperature” means the temperature at which the solvent or solvent system refluxes or boils at atmospheric pressure.

The solution obtained in step-(a) is optionally stirred at a temperature of about 50° C. to the reflux temperature of the solvent used for at least 15 minutes, and specifically at a temperature of about 60° C. to about 90° C. for about 20 minutes to about 2 hours.

The carbon treatment or silica gel treatment in step-(b) is carried out by methods known in the art, for example, by stirring the solution with finely powdered carbon or silica gel at a temperature of below about 80° C. for at least 15 minutes, specifically at a temperature of about 40° C. to about 70° C. for at least 30 minutes; and filtering the resulting mixture through hyflo to obtain a filtrate by removing charcoal or silica gel. Specifically, the finely powdered carbon is an active carbon. A specific mesh size of silica gel is 40-500 mesh, and more specifically 60-120 mesh.

In one embodiment, the isolation of highly pure tapentadol hydrochloride substantially free of impurities in step-(c) is carried out by cooling the solution while stirring at a temperature of below 30° C., specifically at about 0° C. to about 30° C. for about 30 minutes to about 10 hours, and more specifically at about 20° C. to about 30° C. for about 1 hour to about 5 hours.

The recovery of highly pure tapentadol hydrochloride substantially free of impurities in step-(c) is accomplished by techniques such as filtration, filtration under vacuum, decantation, centrifugation, or a combination thereof. In one embodiment, the tapentadol hydrochloride is recovered by filtration employing a filtration media of, for example, a silica gel or celite.

The highly pure tapentadol hydrochloride obtained by the above process may be further dried by the methods as described above.

In another embodiment, the highly pure tapentadol hydrochloride substantially free of at least one, or more, of the isomer-A, isomer-B and isomer-D, and ‘1.09 RRt’, ‘1.14 RRt’, ‘1.24 RRt’, ‘1.47 RRt’ and ‘2.19 RRt’ impurities disclosed herein for use in the pharmaceutical compositions has a D₉₀ particle size of less than or equal to about 400 microns, specifically about 1 micron to about 200 micron, and most specifically about 10 microns to about 100 microns.

In another embodiment, the particle sizes of the highly pure tapentadol hydrochloride substantially free of at least one, or more, of the isomer-A, isomer-B and isomer-D, and ‘1.09 RRt’, ‘1.14 RRt’, ‘1.24 RRt’, ‘1.47 RRt’ and ‘2.19 RRt’ impurities are produced by a mechanical process of reducing the size of particles which includes any one or more of cutting, chipping, crushing, milling, grinding, micronizing, trituration or other particle size reduction methods known in the art, to bring the solid state form to the desired particle size range.

The following examples are given for the purpose of illustrating the present disclosure and should not be considered as limitation on the scope or spirit of the disclosure.

### EXAMPLES

### Example 1

Preparation of Racemic 3-(dimethylamino)-1-(3-hydroxyphenyl)-2-methylpropan-1-one hydrochloride

3-Hydroxy propiophenone (250 g) was taken into a reaction flask, followed by the addition of paraformaldehyde (110 g), N,N-dimethylamine hydrochloride (300 g), isopropyl alcohol (625 ml) and concentrated hydrochloric acid (19.8 ml). The reaction mixture was heated at 85-90° C. for 8 to 9 hours, the resulting mass was cooled to 20-25° C., followed by the addition of water (1250 ml). The resulting aqueous solution was washed with toluene (3×450 ml), the aqueous layer was separated, followed by the addition of aqueous ammonia (200 ml) and then extracting the aqueous solution with dichloromethane (3×500 ml). The organic layers were combined, dried over sodium sulfate (30 g) and then the organic solvent was distilled off under reduced pressure at 30-35° C. to produce an oily mass. Isopropanol (1000 ml) and isopropanolic-HCl (400 ml) were added to the resulting oil and the mixture was stirred for 3 hours at 20-25° C. The resulting solid was filtered, washed with isopropanol (1000 ml) and then the solid was dried in an air oven at 60° C. for 10 to 12 hours to produce 243 g of racemic 3-(dimethylamino)-1-(3-hydroxyphenyl)-2-methylpropan-1-one hydrochloride (Purity by HPLC: 96.76%).

### Example 2

Preparation of Racemic 3-(dimethylamino)-1-(3-hydroxyphenyl)-2-methylpropan-1-one Hydrochloride

3-Hydroxy propiophenone (200 g) was taken into a reaction flask, followed by the addition of paraformaldehyde (87.9 g), N,N-dimethylamine hydrochloride (239 g), isopropyl alcohol (500 ml) and concentrated hydrochloric acid (15 ml). The reaction mixture was heated at 85-90° C. for 8 to 9 hours and the resulting mass was cooled to 20-25° C., followed by the addition of water (1500 ml). The resulting aqueous solution was washed with toluene (2×500 ml), the aqueous layer was separated, followed by the addition of aqueous ammonia (250 ml) and then extracting the aqueous solution with dichloromethane (3×400 ml). The organic layers were combined, dried over sodium sulfate (30 g) and then the organic solvent was distilled off under reduced pressure at 30-35° C. to produce an oily mass. Isopropanol (1000 ml) and concentrated hydrochloric acid (150 ml) were added to the resulting oil at 20-25° C. and the mixture was stirred for 12 hours at 20-25° C. The resulting slurry was cooled to 10° C. and maintained for 1 hour at 10° C. The resulting solid was filtered, washed with isopropanol (250 ml) and then the solid was dried in an air oven at 60° C. for 10 to 12 hours to produce 137 g of racemic 3-(dimethylamino)-1-(3-hydroxyphenyl)-2-methylpropan-1-one hydrochloride (Purity by HPLC: 99.68%).

### Example 3

Preparation of Racemic 3-(dimethylamino)-1-(3-hydroxyphenyl)-2-methylpropan-1-one

Water (500 ml) and aqueous ammonia (100 ml) were added to racemic 3-(dimethylamino)-1-(3-hydroxyphenyl)-2-methylpropan-1-one hydrochloride (100 g, obtained in example 1), and the resulting aqueous reaction mass was extracted with dichloromethane (3×500 ml). The organic layers were combined and then dried over sodium sulfate (25 g). The organic solvent was distilled under reduced pressure at 35-40° C. to give an oily mass (free base).

### Example 4

Preparation of racemic mixture of (2R,3 S)/(2S,3R)-1-(Dimethylamino)-3-(3-hydroxyphenyl)-2-methylpentan-3-ol Hydrochloride

Ethyl magnesium chloride (2M solution, 500 ml) and tetrahydrofuran (600 ml) were taken into a reaction flask, followed by the slow addition of a solution of racemic 3-(dimethylamino)-1-(3-hydroxyphenyl)-2-methylpropan-1-one (obtained in example 3) in tetrahydrofuran (800 ml) at 20-25° C. The reaction mass was stirred for 1 hour at 0-5° C. and then at 20-25° C. An aqueous solution of ammonium chloride (800 ml, 20%) was added to the resulting mass, the organic layer was separated and the aqueous layer was extracted with dichloromethane (800 ml). The organic layers were combined, dried over sodium sulfate (25 g) and then the organic solvent was distilled off under vacuum at 30-40° C. to provide an oily mass. Isopropanol (300 ml) was added to the oil, followed by the addition of IPA-HCl (500 ml) and then stirring the mixture overnight at 20-25° C. The reaction mixture was further cooled to 0 to 5° C. for 2 hours. The solid was filtered, washed with isopropanol (100 ml) and then the solid was dried in an air oven at 55-60° C. for 8 to 9 hours to give 98 g of racemic mixture of (2R,3S)/(2S,3R)-1-(Dimethylamino)-3-(3-hydroxyphenyl)-2-methylpentan-3-ol hydrochloride (Purity by HPLC: 99.67%; Yield: 86.5%).

### Example 5

Preparation of Racemic Mixture of (2R,3 S)/(2S,3R)-1-(Dimethylamino)-3-(3-hydroxyphenyl)-2-methylpentan-3-ol

Water (250 ml) and aqueous ammonia (70 ml) were added to the racemic mixture of (2R,3 S)/(2S, 3R)-1-(Dimethylamino)-3-(3-hydroxyphenyl)-2-methylpentan-3-ol hydrochloride (70 g, obtained in Example 4) and the resulting aqueous reaction mass was extracted with dichloromethane (3×250 ml). The organic layers were combined, dried over sodium sulfate (10 g) and then the organic solvent was distilled off under reduced pressure at 35-40° C. to give an oily mass, which was then slowly solidified (after 3 hours) to produce a racemic mixture of (2R,3 S)/(2S, 3R)-1-(Dimethylamino)-3-(3-hydroxyphenyl)-2-methylpentan-3-ol as a solid (free base).

### Example 6

Resolution of Racemic Mixture of (2R,3 S)/(2S,3R)-1-(Dimethylamino)-3-(3-hydroxyphenyl)-2-methylpentan-3-ol using D-(−)-Tartaric Acid

D-(−)-Tartaric acid (44.3 g) was added to isopropanol (2000 ml) and the slurry was heated at 80-85° C. to form a clear solution. A solution of racemic mixture of (2R,3S)/(2S,3R)-1-(Dimethylamino)-3-(3-hydroxyphenyl)-2-methylpentan-3-ol in isopropanol (500 ml) was slowly added to the solution at 80 to 85° C. and then maintained for 1 hour at 80-85° C. Water (550 ml) was slowly added to the resulting clear solution and then gradually cooled to 20-25° C. and stirred for overnight at 20-25° C. The solid was filtered, washed with isopropanol (150 ml) and then the solid was dried in an air oven at 55-60° C. for 2 hours to produce 38 g of (2S, 3R)-1-(Dimethylamino)-3-(3-hydroxyphenyl)-2-methylpentan-3-ol D-tartrate salt [Purity by HPLC: 99.86%; S.O.R=−14.3° (1% in water)].

### Example 7

Isolation of (2S,3R)-1-(Dimethylamino)-3-(3-hydroxyphenyl)-2-methylpentan-3-ol as a Solid (desired enantiomer)

Water (20 ml) and ammonia (10 ml) were added to (2S,3R)-1-(Dimethylamino)-3-(3-hydroxyphenyl)-2-methylpentan-3-ol D-tartrate salt (1.5 g, obtained in example 6) and the resulting aqueous solution was extracted with dichloromethane (25 ml), followed by distillation of the solvent at 30-35° C. to give (2S,3R)-1-(Dimethylamino)-3-(3-hydroxyphenyl)-2-methylpentan-3-ol as a solid.

### Example 8

Isolation of (2S,3R)-1-(Dimethylamino)-3-(3-hydroxyphenyl)-2-methylpentan-3-ol as an Oil (Desired Enantiomer)

Water (150 ml), aqueous ammonia (30 ml) and dichloromethane (150 ml) were added to (2S, 3R)-1-(Dimethylamino)-3-(3-hydroxyphenyl)-2-methylpentan-3-ol D-tartrate salt (35 g) and the mixture was stirred, followed by separation of the organic layer and then drying over sodium sulfate (15 g). The organic solvent was distilled under reduced pressure at 35-40° C. to give (2S, 3R)-1-(Dimethylamino)-3-(3-hydroxyphenyl)-2-methylpentan-3-ol as an oil [Purity by HPLC: 99.67%; SOR of oil=−21.1° (1% in Methanol)].

### Example 9

Preparation of 3-[(1R,2R)-3-(dimethylamino)-1-ethyl-2-methylpropyl]phenol hydrochloride (Tapentadol Hydrochloride)

2-Methyltetrahydrofuran (320 ml) was added to the (2S,3R)-1-(Dimethylamino)-3-(3-hydroxyphenyl)-2-methylpentan-3-ol (24 g, the oil obtained in example 8) and the solution was cooled to 0-5° C. Trifluoroacetic anhydride (60 ml) was slowly added to the resulting solution and the mixture was stirred for 1 hour at 20-25° C. The reaction mass was transferred into an autoclave, followed by the addition of Pd/C (9 g, 10% Palladium on carbon wet) and then applying hydrogen pressure of 6 Kg. The reaction mass in the autoclave was heated at 50° C. and stirred for 1 hour at 50-55° C. The reaction mass was cooled to 20-25° C., followed by filtration through a hyflow bed. The filtrate was distilled under reduced pressure at 45 to 50° C. to produce an oily residue. Water (150 ml), dichloromethane (150 ml) and aqueous ammonia (30 ml) were added to the residue and the mixture was stirred, followed by the separation of the organic layer. The separated organic layer was dried over sodium sulfate (15 g) and then the organic solvent was distilled under reduced pressure at 35-40° C. to produce an oily mass. Isopropanol (100 ml) was added to the resulting oil, followed by the addition of IPA-HCl (30 ml). The mixture was stirred for 1 hour at 20 to 25° C. and then the slurry was cooled to 0-5° C. for 1 hour. The solid was filtered, washed with isopropanol (30 ml) and then the solid was dried in an air oven at 55 to 60° C. for 10 to 12 hours to produce 15 g of tapentadol hydrochloride [Purity by HPLC: 99.53%; Chiral Purity: 99.9%; and SOR=−25.8° (1% in methanol).

### Example 10

### Purification of Tapentadol Hydrochloride

Isopropyl alcohol (137.5 ml) was added to tapentadol hydrochloride (55 g) at 25-30° C. The resulting mass was heated at 80-85° C. and then methanol (82.5 ml) was added at the same temperature to form a clear solution. The solution was cooled to 25° C. The resulting solid was filtered at 25° C., washed with isopropyl alcohol (55 ml) and then dried in an air oven at 50° C. for 4-5 hours to produce 43 g of pure tapentadol hydrochloride.

### Content of Impurities:

### Before Purification:

Purity by HPLC: 98.96%; Single maximum unknown impurities: ‘1.09 RRt’ impurity: 0.67% and ‘1.47 RRt’ impurity: 0.21%; Chiral Purity: isomer-A: 0.66%, isomer-B: 0.25% and isomer-D: 0.06%.

### After Purification:

Purity by HPLC: 99.81%; Single maximum unknown impurities: ‘1.10 RRt’ impurity: 0.09% and ‘1.24 RRt’ impurity: 0.02%; Chiral Purity: isomer-A: 0.10%, isomer-B: 0.06% and isomer-D: 0.02%.

### Example 11

### Purification of Tapentadol Hydrochloride

Acetonitrile (25 ml) was added to tapentadol hydrochloride (2 g) at 25-30° C. The reaction mass was heated at 80-85° C. and then acetonitrile (85 ml) was added at the same temperature to form a clear solution. The solution was cooled to 40° C. The resulting solid was filtered at 40° C., washed with acetonitrile (5 ml) and then dried in air for 3 hours to produce 1.1 g of pure tapentadol hydrochloride.

### Content of Impurities:

### Before Purification:

Purity by HPLC: 98.2%; Single maximum unknown impurities: ‘1.09 RRt’ impurity: 1.1%, ‘1.14 RRt’ impurity: 0.22% and ‘2.19 RRt’ impurity: 0.17%; Chiral Purity: isomer-A: 1.04%, isomer-B: 0.06% and isomer-D: 0.02%.

### After Purification:

Purity by HPLC: 99.60%; Single maximum unknown impurities: ‘1.10 RRt impurity: 0.22% and '1.24 RRt’ impurity: 0.13%; Chiral Purity: isomer-A: 0.13%, isomer-B: 0.02% and isomer-D: 0.06%.

### Example 12

### Purification of Tapentadol Hydrochloride

Isopropyl alcohol (5 ml) was added to tapentadol hydrochloride (2 g) at 25-30° C. The resulting mass was heated at 80-85° C., followed by the addition of water (0.4 ml) at the same temperature to form a clear solution. The solution was cooled to 25° C., the separated solid was filtered at the same temperature, washed with isopropyl alcohol (2 ml) and then dried in air for 15 hours to produce 1.3 g of pure tapentadol hydrochloride.

### Content of Impurities:

### Before Purification:

Purity by HPLC: 98.2%; Single maximum unknown impurities: ‘1.09 RRt’ impurity: 1.1%, ‘1.14 RRt’ impurity: 0.22% and ‘2.19 RRt’ impurity: 0.17%; Chiral Purity: isomer-A: 1.02%, isomer-B: 0.12% and isomer-D: 0.03%.

### After Purification:

Purity by HPLC: 99.53%; Single maximum unknown impurities: '1.10 RRt impurity: 0.21%; Chiral Purity: isomer-A: 0.20%, isomer-B: 0.06% and isomer-D: Below detection limit.

### Example 13

### Purification of Tapentadol Hydrochloride

Acetonitrile (10 ml) was added to tapentadol hydrochloride (2 g) at 25-30° C. The reaction mass was heated at 80-85° C., followed by the addition of water (0.7 ml) at the same temperature to form clear solution. The solution was cooled at 25° C., the separated solid was filtered at the same temperature, washed with acetonitrile (2 ml) and then dried in an air oven at 60° C. to produce 0.74 g of pure tapentadol hydrochloride.

### Content of Impurities:

### Before Purification:

Purity by HPLC: 98.53%; Single maximum unknown impurities: ‘1.09 RRt’ impurity: 1.05%, ‘1.14 RRt’ impurity: 0.22% and ‘2.19 RRt’ impurity: 0.17%; Chiral Purity: isomer-A: 0.10%, isomer-B: 0.06% and isomer-D: 0.03%.

### After Purification:

Purity by HPLC: 99.65%; Single maximum unknown impurities: ‘1.10 RRt’ impurity: 0.1%; Chiral Purity: isomer-A: 0.10%, isomer-B: 0.06% and isomer-D: Below detection limit.

### Example 14

### Purification of Tapentadol Hydrochloride

Ethanol (3 ml) was added to tapentadol hydrochloride (1 g) at 25-30° C. The reaction mass was heated at 75-80° C., followed by the addition of methanol (0.5ml) at the same temperature to form a clear solution. The solution was cooled to 25° C., the separated solid was filtered at the same temperature, washed with ethanol (2 ml) and then dried in air for 15 hours to produce 0.6 g of pure tapentadol hydrochloride.

### Content of Impurities:

### Before Purification:

Purity by HPLC: 99.04%; Single maximum unknown impurities: ‘1.09 RRt’ impurity: 0.62% and ‘1.46 RRt’ impurity: 0.63%; Chiral Purity: isomer-A: 0.56%, isomer-B: 0.29% and isomer-D: 0.06%.

### After Purification:

Purity by HPLC: 99.86%; Single maximum unknown impurities: ‘1.10 RRt’ impurity: 0.07%; Chiral Purity: isomer-A: 0.07%, isomer-B: 0.05% and isomer-D: Below detection limit.

As used herein, the term, “detectable” refers to a measurable quantity measured using an HPLC method having a detection limit of 0.01 area-%.

As used herein, in connection with amount of impurities in tapentadol hydrochloride, the term “not detectable” means not detected by the HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “gm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A process for preparing tapentadol, 3-[(1R,2R)-3-(dimethylamino)-1-ethyl-2-methylpropyl]phenol, of formula I: 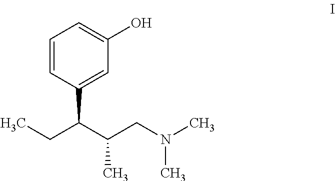 [SMILES: CC[C@@H](c1cccc(O)c1)[C@@H](C)CN(C)C]
or a pharmaceutically acceptable salt thereof, comprising
a) reacting 3-hydroxypropiophenone of formula V: 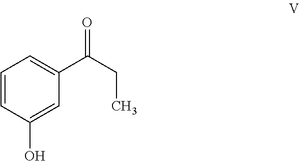
with N,N-dimethylamine hydrochloride in the presence of para-formaldehyde in a first solvent to produce a racemic 3-(dimethylamino)-1-(3-hydroxyphenyl)-2-methylpropan-1-one of formula IV: 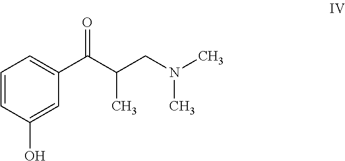
or an acid addition salt thereof;
b) reacting the compound of formula IV or the acid addition salt thereof with an ethyl magnesium halide in a second solvent to produce a racemic mixture of (2R,3 S)/(2S,3R)-1-(Dimethylamino)-3-(3-hydroxyphenyl)-2-methylpentan-3-ol of formula III: 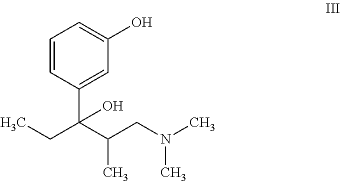 [SMILES: CCC(O)(c1cccc(O)c1)C(C)CN(C)C]
or an acid addition salt thereof;
c) treating the racemic mixture obtained in step-(b) with an optically active acid in a third solvent to produce a diastereomeric mixture containing a diastereomeric excess of a desired diastereomeric salt;
d) separating the desired diastereomeric salt from the diastereomeric mixture obtained in step-(c);
e) neutralizing the desired diastereomeric salt obtained in step-(d) with a base in a fourth solvent to produce an enantiomerically pure (2S,3R)-1-(Dimethylamino)-3-(3-hydroxyphenyl)-2-methylpentan-3-ol of formula II: 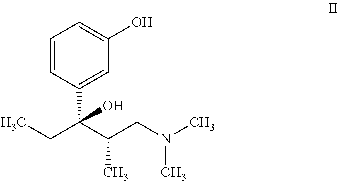 [SMILES: CC[C@](O)(c1cccc(O)c1)[C@@H](C)CN(C)C]
and optionally converting the enantiomerically pure compound of formula II obtained into an acid addition salt thereof; and
f) reacting the enantiomerically pure compound of formula II or the acid addition salt thereof obtained in step-(e) with trifluoroacetic anhydride in a fifth solvent to produce a reaction mass, followed by hydrogenating the reaction mass in the presence of a hydrogenation catalyst to produce the tapentadol of formula I, and optionally converting the tapentadol of formula I obtained into a pharmaceutically acceptable salt thereof.

2. The process of claim 1, wherein the first solvent used in step-(a) is selected from the group consisting of water, an alcohol, an ether, a hydrocarbon, a chlorinated hydrocarbon, a nitrile solvent, a polar aprotic solvent, and mixtures thereof; wherein the second solvent used in step-(b) is selected from the group consisting of a ketone, a cyclic ether, an aliphatic ether, and mixtures thereof; wherein the third solvent used in step-(c) is selected from the group consisting of water, an alcohol, a ketone, an ester, a hydrocarbon, a chlorinated hydrocarbon, a polar aprotic solvent, and mixtures thereof; wherein the fourth solvent used in step-(e) is selected from the group consisting of water, an alcohol, a ketone, a cyclic ether, an aliphatic ether, a hydrocarbon, a chlorinated hydrocarbon, a nitrile, an ester, and mixtures thereof; and wherein the fifth solvent used in step-(f) is selected from the group consisting of water, an alcohol, a ketone, a cyclic ether, an aliphatic ether, a hydrocarbon, a chlorinated hydrocarbon, a nitrile solvent, and mixtures thereof

3. The process of claim 2, wherein the first solvent used in step-(a) is selected from the group consisting of methanol, ethanol, isopropanol, n-butanol, isobutanol, tert-butanol, amyl alcohol, acetonitrile, and mixtures thereof; wherein the second solvent used in step-(b) is selected from the group consisting of acetone, methyl ethyl ketone, methyl isobutyl ketone, methyl tert-butyl ketone, tetrahydrofuran, 2-methyl tetrahydrofuran, dioxane, diethyl ether, diisopropyl ether, monoglyme, diglyme, and mixtures thereof; wherein the third solvent used in step-(c) is selected from the group consisting of water, methanol, ethanol, isopropanol, and mixtures thereof; wherein the fourth solvent used in step-(e) is selected from the group consisting of water, methylene chloride, n-hexane, n-heptane, cyclohexane, toluene, xylene, and mixtures thereof; and wherein the fifth solvent used in step-(f) is selected from the group consisting of methanol, ethanol, isopropanol, acetonitrile, tetrahydrofuran, 2-methyl tetrahydrofuran, and mixtures thereof.

4. The process of claim 1, wherein the reaction in step-(a) is carried out at a temperature of 0° C. to the reflux temperature of the solvent used for at least 4 hours;
wherein the reaction in step-(b) is carried out at a temperature of below about 60° C. for at least 30 minutes; wherein the reaction in step-(c) is carried out at a temperature of 0° C. to the reflux temperature of the solvent used for at least 30 minutes; wherein the pH of the reaction mass in step-(e) is adjusted to above 7; wherein the reaction in step-(f) is carried out at a temperature of −20° C. to the reflux temperature of the solvent used for at least 10 minutes; and wherein the hydrogenation reaction in step-(f) is carried out at a temperature of 0° C. to the reflux temperature of the solvent used for at least 30 minutes.

5. The process of claim 4, wherein the reaction in step-(a) is carried out at the reflux temperature of the solvent used for about 8 hours to about 12 hours; wherein the reaction in step-(b) is carried out at a temperature of about 0° C. to about 25° C. for about 1 hour to about 10 hours; wherein the reaction in step-(c) is carried out at a temperature of about 50° C. to about 100° C. for about 30 minutes to about 10 hours; wherein the pH of the reaction mass in step-(e) is adjusted between 7 and 8; wherein the reaction in step-(f) is carried out at a temperature of about 0° C. to about 50° C. for about 20 minutes to about 6 hours; and wherein the hydrogenation reaction in step-(f) is carried out at a temperature of about 20° C. to about 65° C. for about 45 minutes to about 7 hours.

6. The process of claim 1, wherein the N,N-dimethylamine hydrochloride in step-(a) is used in a ratio of about 1 to 4 equivalents with respect to the 3-hydroxypropiophenone of formula V; wherein the ethyl magnesium halide used in step-(b) is selected from the group consisting of ethyl magnesium chloride, ethyl magnesium bromide and ethyl magnesium iodide; wherein the ethyl magnesium halide in step-(b) is used in a ratio of about 2 to 20 equivalents with respect to the racemic 3-(dimethylamino)-1-(3-hydroxyphenyl)-2-methylpropan-1-one formula IV; wherein the optically active acid used in step-(c) is selected from the group consisting of D-(−)-tartaric acid, L-(+)-tartaric acid, S-naproxen, (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof; wherein the resolving agent in step-(c) is used in a ratio of about 1 to 5 equivalents with respect to the racemic mixture of (2R,3 S)/(2S,3R)-1-(Dimethylamino)-3-(3-hydroxyphenyl)-2-methylpentan-3-ol of formula III; wherein the base used in step-(e) is an organic or inorganic base; and wherein the hydrogenation catalyst used in step-(f) is selected from the group consisting of palladium hydroxide, palladium on carbon, platinum on carbon, platinum oxide, rhodium on carbon, and rhodium on alumina.

7. The process of claim 6, wherein the N,N-dimethylamine hydrochloride in step-(a) is used in a ratio of about 2 to 2.5 equivalents with respect to the 3-hydroxypropiophenone of formula V; wherein the ethyl magnesium halide used in step-(b) is ethyl magnesium chloride; wherein the ethyl magnesium halide in step-(b) is used in a ratio of about 3 to 10 equivalents with respect to the racemic 3-(dimethylamino)-1-(3-hydroxyphenyl)-2-methylpropan-1-one formula IV; wherein the optically active acid used in step-(c) is D-(−)-tartaric acid; wherein the resolving agent in step-(c) is used in a ratio of about 1 to 1.5 equivalents with respect to the racemic mixture of (2R,3S)/(2S,3R)-1-(Dimethylamino)-3-(3-hydroxyphenyl)-2-methylpentan-3-ol of formula III; wherein the base used in step-(e) is selected from the group consisting of triethylamine, trimethylamine, dimethyl amine, tert-butyl amine, aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide; and wherein the hydrogenation catalyst used in step-(f) is palladium on carbon.

8. Solid state form of a tapentadol intermediate selected from a racemic 3-(dimethylamino)-1-(3-hydroxyphenyl)-2-methylpropan-1-one hydrochloride salt, a racemic mixture of (2R,3 S)/(2S,3R)-1-(Dimethylamino)-3-(3-hydroxyphenyl)-2-methylpentan-3-ol, a racemic mixture of (2R,3 S)/(2S,3R)-1-(Dimethylamino)-3-(3-hydroxyphenyl)-2-methylpentan-3-ol hydrochloride salt, (2S,3R)-1-(Dimethylamino)-3-(3-hydroxyphenyl)-2-methylpentan-3-ol D-tartrate salt and (2S,3R)-1-(Dimethylamino)-3-(3-hydroxyphenyl)-2-methylpentan-3-ol, wherein:
a) the solid state form of the racemic 3-(dimethylamino)-1-(3-hydroxyphenyl)-2-methylpropan-1-one hydrochloride salt is a crystalline form characterized by the following properties:
i) a powder X-ray diffraction pattern having peaks at about 6.38, 7.34, 11.68, 13.81, 15.52, 16.9, 17.42, 17.67, 18.14, 18.51, 19.20, 20.47, 20.70, 20.96, 21.23, 21.58, 22.20, 23.55, 23.80, 24.16, 24.85, 25.53, 25.77, 26.31, 26.85, 27.39, 29.09, 30.99, 32.49 and 33.76±0.2 degrees 2-theta substantially in accordance with FIG. 1;
ii) a powder X-ray diffraction pattern having peaks at about 6.42, 7.38, 11.71, 15.57, 18.19, 18.55, 19.26, 20.51, 20.77, 21.0, 22.25, 23.59, 23.85, 24.20, 25.81, 26.35, 26.87, 32.54 and 33.81±0.2 degrees 2-theta substantially in accordance with FIG. 2;
b) the solid state form of the racemic mixture of (2R,3S)/(2S,3R)-1-(Dimethylamino)-3-(3-hydroxyphenyl)-2-methylpentan-3-ol is a crystalline form characterized by a powder X-ray diffraction pattern having peaks at about 12.1, 12.51, 13.13, 14.16, 14.7, 15.78, 17.25, 19.09, 21.28, 21.69, 22.01, 23.10, 23.4 and 24.36±0.2 degrees 2-theta substantially in accordance with FIG. 3;
c) the solid state form of the racemic mixture of (2R,3S)/(2S,3R)-1-(Dimethylamino)-3-(3-hydroxyphenyl)-2-methylpentan-3-ol hydrochloride salt is a crystalline form characterized by a powder X-ray diffraction pattern having peaks at about 8.11, 9.27, 10.92, 13.30, 13.64, 14.62, 15.30, 18.32, 18.66, 19.65, 20.51, 20.90, 23.42, 23.93, 24.41, 25.25, 25.80, 27.32, 27.52, 29.59, 29.91, 30.22, 30.88, 31.63, 32.26, 35.34 and 36.43±0.2 degrees 2-theta substantially in accordance with FIG. 4;
d) the solid state form of the (2S,3R)-1-(Dimethylamino)-3-(3-hydroxyphenyl)-2-methylpentan-3-ol D-tartrate salt is a crystalline form characterized by a powder X-ray diffraction pattern having peaks at about 5.78, 11.55, 15.86, 16.14, 17.35, 18.33, 18.88, 20.48, 21.24, 21.81, 23.17, 23.77, 28.59 and 29.07±0.2 degrees 2-theta substantially in accordance with FIG. 5; and
e) the solid state form of the (2S,3R)-1-(Dimethylamino)-3-(3-hydroxyphenyl)-2-methylpentan-3-ol is a crystalline form characterized by a powder X-ray diffraction pattern having peaks at about 9.44, 11.25, 12.35, 12.83, 14.27, 15.6, 16.34, 17.85, 18.93, 21.42, 21.81, 22.63, 23.48, 24.91 and 25.92±0.2 degrees 2-theta substantially in accordance with FIG. 6.

9-11. (canceled)

12. A purification process for obtaining highly pure tapentadol hydrochloride substantially free of impurities of claim 9, comprising:
a) providing a solution of tapentadol hydrochloride in a solvent or a solvent mixture at a temperature of above 40° C., wherein the solvent or the solvent mixture is selected from the group consisting of water, methanol, ethanol, isopropyl alcohol, acetonitrile, and mixtures thereof;
b) optionally, subjecting the solution obtained in step-(a) to carbon treatment or silica gel treatment; and
c) isolating and recovering the highly pure tapentadol hydrochloride substantially free of impurities from the solution obtained in step-(a) or step-(b), wherein the isolation is carried out by cooling the solution at a temperature of below 40° C.,
wherein the highly pure tapentadol hydrochloride substantially free of impurities has one or more of a 3-[(1R,2S)-3-(dimethylamino)-1-ethyl-2-methylpropyl]phenol hydrochloride impurity (isomer-A), a 3-[(1S,2R)-3-(dimethylamino)-1-ethyl-2-methylpropyl]phenol hydrochloride impurity (isomer-B) impurity, a 3-[(1S,2S)-3-(dimethylamino)-1-ethyl-2-methylpropyl]phenol hydrochloride impurity (isomer-D), a ‘1.09 RRt’ impurity, a ‘1.14 RRt’ impurity, a ‘1.24 RRt’ impurity, a ‘1.47 RRt’ impurity and a ‘2.19 RRt’ impurity in an amount of less than about 0.15 area-% as measured by HPLC.

13. The process of claim 12, wherein the solution in step-(a) is provided by dissolving tapentadol hydrochloride in the solvent or the solvent mixture at a temperature of about 50° C. to the reflux temperature of the solvent used; wherein the isolation of highly pure tapentadol hydrochloride substantially free of impurities in step-(c) is carried out by cooling the solution while stirring at a temperature of below 30° C.; and wherein the recovering in step-(c) is carried out by filtration, filtration under vacuum, decantation, centrifugation, filtration employing a filtration media of a silica gel or celite, or a combination thereof.

14. The process of claim 13, wherein the solution in step-(a) is provided by dissolving tapentadol hydrochloride in the solvent or the solvent mixture at a temperature of about 60° C. to about 90° C.; and wherein the isolation in step-(c) is carried out by cooling the solution while stirring at a temperature of about 0° C. to about 30° C. for about 30 minutes to about 10 hours.

15. The process of claim 12 further comprising reducing the particle size of the highly pure tapentadol hydrochloride substantially free of impurities to a D₉₀ particle size of less than or equal to about 400 microns.

16. The process of claim 15, wherein the D₉₀ particle size is about 1 micron to about 200 microns.

17. The process of claim 12, wherein the highly pure tapentadol hydrochloride substantially free of impurities comprises one or more of the isomer-A, isomer-B, isomer-D, and the ‘1.09 RRt’, ‘1.14 RRt’, ‘1.24 RRt’, ‘1.47 RRt’ and ‘2.19 RRt’ impurities each in an amount of less than about 0.05 area-%.

18. The process of claim 12, wherein the highly pure tapentadol hydrochloride substantially free of impurities has a non-detectable amount of one or more of the isomer-A, isomer-B, isomer-D, and the ‘1.09 RRt’, ‘1.14 RRt’, ‘1.24 RRt’, ‘1.47 RRt’ and ‘2.19 RRt’ impurities as measured by HPLC.

19. The process of claim 16, wherein reducing the particle size of the highly pure tapentadol hydrochloride substantially free of impurities comprises cutting, chipping, crushing, milling, grinding, micronizing, or trituration.

20. The process of claim 12, wherein the highly pure tapentadol hydrochloride substantially free of impurities is greater than 99% pure.

21. The process of claim 1, wherein the hydrogenation catalyst of step f) is used in the ratio of about 0.05% (w/w) to 10% (w/w) with respect to (−)-3-[(1R,2R)-3-(dimethylamino)-1-ethyl-1-hydroxy-2-methylpropyl]phenol of formula II.

22. The process of claim 1, wherein the pharmaceutically acceptable salt of tapentadol in step f) is selected from hydrochloride, hydrobromide, oxalate, nitrate, sulphate, phosphate, fumarate, succinate, maleate, besylate, tosylate, palmitate and tartrate.

23. The process of claim 1, wherein the pharmaceutically acceptable salt of tapentadol in step f) is a hydrochloride salt.
